(19) United States
(12) Patent Application Publication
(10) Pub. No.: US 2025/0332480 A1
(43) Pub. Date: Oct. 30, 2025

(54) EXTREMITY REHABILITATION METHOD AND ROBOTIC DEVICE USING THE SAME

(71) Applicant: Futronics (NA) Corporation, Pasadena, CA (US)

(72) Inventors: FANGYUN ZHAO, Pasadena, CA (US); Yunfei Guo, Pasadena, CA (US); Weide Zhang, Pasadena, CA (US); Wenda Xu, Pasadena, CA (US); Fulan Li, Pasadena, CA (US); Chengkun Zhang, Pasadena, CA (US)

(21) Appl. No.: 18/648,264

(22) Filed: Apr. 26, 2024

(51) Int. Cl. A63B 24/00; G16H 20/30

(52) U.S. Cl. CPC A63B 24/0075; G16H 20/30

(57) ABSTRACT

Extremity rehabilitation using a robotic device is disclosed. An extremity rehabilitation method assists a user to perform extremity rehabilitation by: receiving, from a database, personalized information of the user, where the personalized information is generated based on at least a real-life routine of the user; generating, based on the personalized information, a customized routine of an extremity rehabilitation process; determining a scene of the extremity rehabilitation process; conducting the extremity rehabilitation process by, according to the customized routine, controlling a robotic device to perform interactions with the user and displaying at least an object of the interactions in the scene through the display device; and generating, based on a result of the interactions, a personalized report for the user.

### TECHNICAL FIELD

The present disclosure relates to rehabilitation technology, and particularly to an extremity rehabilitation method and a robotic device using the same.

### BACKGROUND

Benefiting from the rapid development of computer hardware and the maturity of artificial intelligence (AI) techniques, modern robotic devices such as robotic arms and mobile robots have been used in various scenes of human's daily life to provide various services such as healthcare and housework. As to the healthcare industry, because game-based sessions have recently been proven to be more effective in sensorimotor recovery than traditional therapy sessions, game-based rehabilitation interventions and robotic therapies have been used to improve motivations in rehab activities and stroke recovery.

Traditional interventions merely focus on physical rehabilitation and motor learning for recovery with gamification, while some illnesses like stroke do not just involve physical injury. The physical constraints were a result of disruption in the neural system, and typical stroke patients also show symptoms of cognitive dysfunction such as dementia. Even if research has shown that gamification and computerized programs can benefit stroke patients' cognitive recovery, there is still none of the robotic therapies or gamification interventions that have considered combining physical and cognitive therapies in single sessions.

### BRIEF DESCRIPTION OF DRAWINGS

In order to more clearly illustrate the technical solutions in this embodiment, the drawings used in the embodiments or the description of the prior art will be briefly introduced below. In the drawing(s), like reference numerals designate corresponding parts throughout the figures. It should be understood that, the drawings in the following description are only examples of the present disclosure. For those skilled in the art, other drawings can be obtained based on these drawings without creative works.

FIG. 1 is a schematic diagram of a scenario of extremity rehabilitation using a robotic device and a display device according to some embodiments of the present disclosure.

FIG. 2 is a schematic block diagram illustrating a healthcare system according to some embodiments of the present disclosure.

FIG. 3 is a schematic block diagram illustrating a robotic device of the healthcare system of FIG. 2.

FIG. 4 is a schematic block diagram of an example of performing upper limb rehabilitation using the robotic device of FIG. 3.

FIG. 5 is a flow chart of an example of generating a customized routine of an extremity rehabilitation process according to some embodiments of the present disclosure.

FIG. 6 is a schematic diagram of displaying a scene of a wake-up routine through the display device of the healthcare system of FIG. 2.

FIG. 7 is a flow chart of an example of an initialization process of the extremity rehabilitation process according to some embodiments of the present disclosure.

FIG. 8 is a flow chart of an example of displaying object of the interactions in a scene through the display device of the healthcare system of FIG. 2 according to some embodiments of the present disclosure.

FIG. 9 is a flow chart of an example of controlling the robotic device of FIG. 3 to perform interactions with the user to generating a personalized report according to some embodiments of the present disclosure.

FIG. 10 is a flow chart of an example of conducting the extremity rehabilitation process in augmented reality manner according to some embodiments of the present disclosure.

FIG. 11 is a schematic diagram of displaying the result of the example of FIG. 10 that conducts the extremity rehabilitation process in augmented reality manner through the display device of the healthcare system of FIG. 2.

FIG. 12 is a schematic diagram of displaying result of the example of FIG. 9 that generates the personalized report for the user through the display device of the healthcare system of FIG. 2.

### DETAILED DESCRIPTION

In order to make the objects, features and advantages of the present disclosure more obvious and easy to understand, the technical solutions in this embodiment will be clearly and completely described below with reference to the drawings. Apparently, the described embodiments are part of the embodiments of the present disclosure, not all of the embodiments. All other embodiments obtained by those skilled in the art based on the embodiments of the present disclosure without creative efforts are within the scope of the present disclosure.

It is to be understood that, when used in the description and the appended claims of the present disclosure, the terms “including”, “comprising”, “having” and their variations indicate the presence of stated features, integers, steps, operations, elements and/or components, but do not preclude the presence or addition of one or a plurality of other features, integers, steps, operations, elements, components and/or combinations thereof.

It is also to be understood that, the terminology used in the description of the present disclosure is only for the purpose of describing particular embodiments and is not intended to limit the present disclosure. As used in the description and the appended claims of the present disclosure, the singular forms “one”, “a”, and “the” are intended to include the plural forms as well, unless the context clearly indicates otherwise.

It is also to be further understood that the term “and/or” used in the description and the appended claims of the present disclosure refers to any combination of one or more of the associated listed items and all possible combinations, and includes such combinations.

In the present disclosure, the terms “first”, “second”, and “third” are for descriptive purposes only, and are not to be comprehended as indicating or implying the relative importance or implicitly indicating the amount of technical features indicated. Thus, the feature limited by “first”, “second”, and “third” may include at least one of the feature either explicitly or implicitly. In the description of the present disclosure, the meaning of “a plurality” is at least two, for example, two, three, and the like, unless specifically defined otherwise.

In the present disclosure, the descriptions of “one embodiment”, “some embodiments” or the like described in the specification mean that one or more embodiments of the present disclosure can include particular features, structures, or characteristics which are related to the descriptions of the descripted embodiments. Therefore, the sentences “in one embodiment”, “in some embodiments”, “in other embodiments”, “in other embodiments” and the like that appear in different places of the specification do not mean that descripted embodiments should be referred by all other embodiments, but instead be referred by “one or more but not all other embodiments” unless otherwise specifically emphasized.

The present disclosure relates to realize extremity rehabilitation through a robotic device. As used herein, the term “robotic device” refers to a machine such as a robotic arm or a mobile robot that includes mechanical components, logic circuitry, computing components, software and/or other specialized components that desired and/or measured force, torque, position, orientation, velocity, and/or angular velocity information is processed by a computing source and such computing source is used to control the force, torque, position, orientation, velocity, angular velocity, and/or physical configuration of the device. The term “sensor” refers to a device, module, machine, or subsystem such as ambient light sensor and image sensor (e.g., camera) whose purpose is to detect events or changes in its environment and send the information to other electronics (e.g., processor).

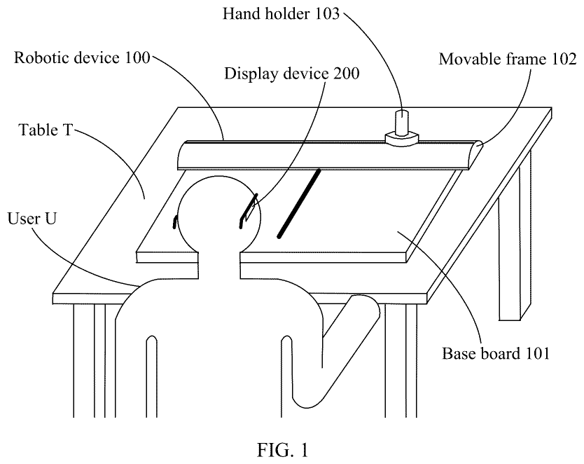

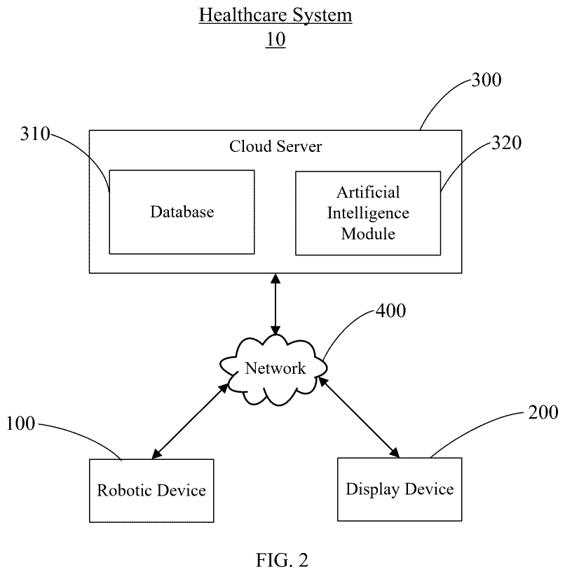

FIG. 1 is a schematic diagram of a scenario of extremity rehabilitation using a robotic device 100 and a display device 200 according to some embodiments of the present; and FIG. 2 is a schematic block diagram illustrating a healthcare system 10 according to some embodiments of the present disclosure. In the scenario (e.g., nursing home, hospital, and home) for a user U (e.g., a patient needs rehabilitation because of an illness such as a stroke) to perform rehab activities, for achieving the purpose of combining physical and cognitive therapies in single sessions, rehabilitation equipment like the robotic device 100 (and auxiliary equipment like the display device 200) may be provided to realize physical therapies, and a system such as the healthcare system 10 that supports the operation of the rehabilitation equipment (and the auxiliary equipment) may be provided to realize cognitive therapies.

In some embodiments, the robotic device 100 is an upper limb rehabilitation robot that can provide rehabilitation-related functions by putting on a table T or other suitable supporter for the user U to operate in a suitable posture like sitting to perform upper limb rehabilitation activities, which may include a base board 101, a movable frame 102 mounted on the base board 101 in a movable manner such as slidable in a y-axis direction (relative to the plane of the base board 101) along a rail of the base board 101, and a hand holder 103 mounted on the movable frame 102 in a movable manner such as slidable in an x-axis direction (relative to the plane of the base board 101) along a rail of the movable frame 102. The hand holder 103 is for supporting an upper limb of the user U. When the user U operates the robotic device 100 through the hand holder 103, the upper limb is moved upon the base board 101 with the movement of the hand holder 103, thereby performing the upper limb rehabilitation activities. The robotic device 100 detects forces from the user U through force sensor, and it may also provide force feedbacks corresponding to the detected forces to simulate real-world physical touch by way of, for example, motorized motion or resistance. The display device 200 is a headset that facilitates the user U to perform the upper limb rehabilitation activities by, for example, displaying related virtual reality (VR)/augmented reality (AR) images, or providing related textual/audio/graphical instructions, introductions, suggestions, or the like, which may include a stereoscopic display to provide separate images for each eye of the user U, a stereo, and sensor(s) like a camera for capturing images of the surroundings of the user U, or an accelerometer and a gyroscope for tracking the pose of the head of the user U to match the orientation of a virtual camera with the positions of the eyes of the user U in the real world so as to simulate the physical presence of the user U in a virtual environment so that the user U is able to look around the artificial world, move around in it, and interact with virtual features or items. The display device 200 may provide different display modes such as a real mode, an AR mode, and a VR mode for the user U to switch through, for example, physical button(s) or remote control of the display device 200.

In other embodiments, the robotic device 100 may be a gantry arm rehabilitation device, a powered exoskeleton, or other robotic device that can provide the same and/or other rehabilitation-related functions to the user U, or be a mobile robot that can provide the same and/or other rehabilitation-related functions and can be navigated in its environment so as to perform, for example, nursing tasks such as physical assessment, wound care, mobility assistance, and emergency response, while on-floor obstacles like falling object, garbage, furniture, pet or the like can be detected to prevent its movement from being affected, for example, obstructed, decelerated, tripped, or slipped, or to avoid dangerous situations like collisions and falling down. The display device 200 may be other display device like a display screen or a multi-projected environment to generate realistic images. In addition, other feedbacks like haptic feedback may be provided through the robotic device 100 or other devices like joysticks.

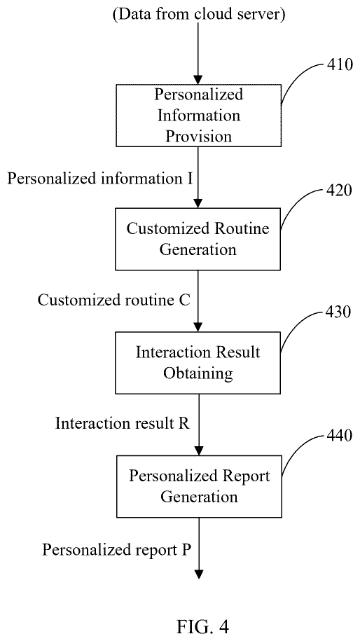

As shown in FIG. 2, in some embodiments, the healthcare system 10 includes the robotic device 100, the display device 200, and a cloud server 300 that communicate over a network 400 which may include, for example, the Internet, intranet, extranet, local area network (LAN), wide area network (WAN), wired network, wireless networks (e.g., Wi-Fi network, Bluetooth network, and mobile network), or other suitable networks, or any combination of two or more such network. The cloud server 300 includes a database 310 and an artificial intelligence module 320. For providing the cognitive therapies to combine with the physical therapies that are provided through the robotic device 100 and the display device 200 in single sessions, the cloud server 300 stores therapy-related data in the database 310, and provides the therapy-related data in response to the request from the robotic device 100 or the display device 200. The therapy-related data includes personalized information I (e.g., name, age, gender, mailing address, email address, and phone number, see FIG. 4)) of users including the user U, and it may include other information of the users such as rehabilitation information and medical records of the users. The database 310 may be an organized collection of structured data including the therapy-related data, which may be controlled by a database management system (DBMS, e.g., MySQL, Microsoft Access, and Oracle Database). The artificial intelligence module 320 may be a software module (of the operation system of the cloud server 300), which performs AI-related functions while AI-related data such as training data and machine learning models may be stored in the database 310 (or the artificial intelligence module 320 itself). The AI-related functions may include computer-enhanced learning, reasoning, and perception, for example, processing external data (e.g., medical records of patients and images of different scenes) received from other device outside the cloud server 300 or processing other information in the therapy-related data (e.g., rehabilitation information and medical records of the users) to obtain information of individual patients and store as the personalized information I of the user U in the database 310 or provide in response to the request from the robotic device 100 or the display device 200. In other embodiments, the healthcare system 10 may include a plurality of robotic devices, for example, the robotic device 100 that is the gantry arm rehabilitation device, another robotic device that is the powered exoskeleton, and the mobile robot so as to meet the requirement of different rehabilitation activities (e.g., activities for upper limb and that for lower limb).

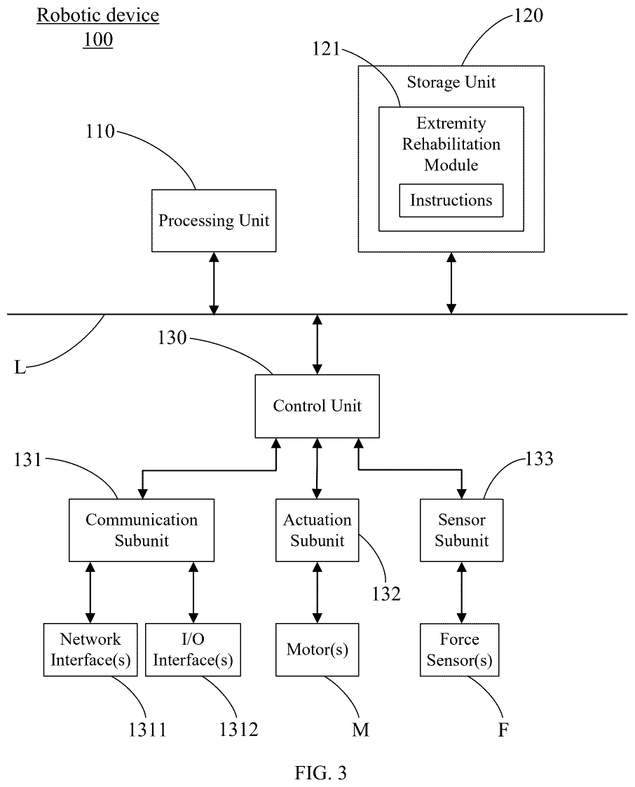

FIG. 3 is a schematic block diagram illustrating the robotic device 100 of the healthcare system 10 of FIG. 2. The robotic device 100 may include a processing unit 110, a storage unit 120, and a control unit 110 that communicate over one or more communication buses or signal lines L. It should be noted that, the robotic device 100 is only one example of robotic device, and the robotic device 100 may have more or fewer components (e.g., unit, subunits, and modules) than shown in above or below, may combine two or more components, or may have a different configuration or arrangement of the components. The processing unit 110 executes various (sets of) instructions stored in the storage unit 120 that may be in form of software programs to perform various functions for the robotic device 100 and to process related data, which may include one or more processors (e.g., CPU). The storage unit 120 may include one or more memories (e.g., high-speed random access memory (RAM) and non-transitory memory), one or more memory controllers, and one or more non-transitory computer readable storage mediums (e.g., solid-state drive (SSD) or hard disk drive). The control unit 110 may include various controllers (e.g., network interface controller, display controller, and physical button controller) and peripherals interface for coupling the input and output peripheral of the robotic device 100, for example, external port (e.g., USB), wireless communication circuit (e.g., RF communication circuit), audio circuit (e.g., speaker circuit), sensor (e.g., inertial measurement unit (IMU)), and the like, to the processing unit 110 and the storage unit 120. In some embodiments, the storage unit 120 may include an extremity rehabilitation module 121 for implementing upper limb rehabilitation functions related to the above-mentioned upper limb rehabilitation activities (e.g., mechanical/electronical functions to control the robotic device 100 so as to enable the user U to perform the upper limb rehabilitation activities), which may be stored in the one or more memories (and the one or more non-transitory computer readable storage mediums. The extremity rehabilitation module 121 may be a software module (of the operation system of the robotic device 100), which has instructions (e.g., instruction for actuating motor(s) M of the robotic device 100) for implementing the above-mentioned upper limb rehabilitation functions. In other embodiments, when the robotic device 100 is the mobile robot, the storage unit 120 may further include a navigation module for implementing navigation functions (e.g., map building and path planning) related to the navigation (and path planning) of the robotic device 100.

The robotic device 100 may further include a communication subunit 131 and an actuation subunit 132. The communication subunit 131 and the actuation subunit 132 communicate with the control unit 110 over one or more communication buses or signal lines that may be the same or at least partially different from the above-mentioned one or more communication buses or signal lines L. The communication subunit 131 is coupled to communication interfaces of the robotic device 100, for example, network interface(s) 1311 for the robotic device 100 to communicate with the display device 200 and the cloud server 300 via network(s), I/O interface(s) 1312 (e.g., a physical button), and the like. The actuation subunit 132 is coupled to component(s)/device(s) for implementing the motions of the robotic device 100 by, for example, actuating motor(s) M of joints of the robotic device 100. The communication subunit 131 may include controllers for the above-mentioned communication interfaces of the robotic device 100, and the actuation subunit 132 may include controller(s) for the above-mentioned component(s)/device(s) for implementing the motions of the robotic device 100. In other embodiments, the communication subunit 131 and/or actuation subunit 132 may just abstract component for representing the logical relationships between the components of the robotic device 100.

The robotic device 100 may further include a sensor subunit 133 which may include a set of sensor(s) and related controller(s), for example, force sensor(s) F, for detecting forces from the user U. The sensor subunit 133 communicates with the control unit 110 over one or more communication buses or signal lines that may be the same or at least partially different from the above-mentioned one or more communication buses or signal lines L. In addition, the sensor subunit 133 may further include a camera and an IMU (inertial measurement unit) (or an accelerometer and a gyroscope), for detecting the situation of the user U to facilitate the above-mentioned upper limb rehabilitation functions. In some embodiments, the various components shown in FIG. 3 may be implemented in hardware, software or a combination of both hardware and software. Two or more of the processing unit 110, the storage unit 120, the control unit 110, the extremity rehabilitation module 121, and other units/subunits/modules may be implemented on a single chip or a circuit. In other embodiments, the sensor subunit 133 may just abstract component for representing the logical relationships between the components of the robotic device 100. In addition, at least a part of them may be implemented on separate chips or circuits.

FIG. 4 is a schematic block diagram of an example of performing upper limb rehabilitation using the robotic device 100 of FIG. 3. In some embodiments, an upper limb rehabilitation method is implemented in the robotic device 100 to, with the auxiliary of the display device 200, assist the user U to perform the upper limb rehabilitation activities by, for example, storing (sets of) instructions corresponding to the upper limb rehabilitation method (e.g., instructions for controlling the motor M(s)) as the extremity rehabilitation module 121 in the storage unit 120 and executing the stored instructions through the processing unit 110, and then the robotic device 100 may be controlled accordingly. The upper limb rehabilitation method may be performed in response to actuating the robotic device 100 through, for example, physical button(s) or a remote control of the robotic device 100 or the display device 200. In other embodiments, the above-mentioned upper limb rehabilitation method may also be performed in response to a request from, for example, (the operation system of) the robotic device 100, the display device 200, or other device of the healthcare system 10.

According to the upper limb rehabilitation method, the processing unit 110 may provide the personalized information I of the user U based on data (e.g., the personalized information I of the user U that is stored in the database 310 or the above-mentioned therapy-related data related to the user U) from the cloud server 300 of the healthcare system 10 (block 410 of FIG. 4). For providing cognitive therapies by supporting the operation of the robotic device 100 (with the auxiliary of the display device 200) based on the personalized information I, the personalized information I has to be generated or enhanced based on real-life routine(s) F (not shown) of the user U. Each real-life routine F of the user U corresponds to a routine in the real-life of the user U like a wake-up routine, dining routine, a toileting routine, or a dressing routine, which includes a plurality of real-life items related to the real-life of the user U. The real-life items of each real-life routine F may be selected from daily living activities (e.g., personal hygiene, dining, toileting, and dressing) and/or other information related to the real-life of the user U like personal habits (e.g., habitual actions, favorite sports, and hobbies), of the user U which may be provide manually (by, for example, the user U or the related medical personnel accessing the cloud server 300) and/or obtain automatically (by, for example, the artificial intelligence module 320 through machine learning means based on the above-mentioned therapy-related data related to the user U) to store in the database 310 of the cloud server 300, and similarly may be selected manually and/or automatically as the real-life items according to, for example, their frequencies to be performed by the user U during a specific period (e.g., the last month).

In some embodiments, the processing unit 110 may provide the personalized information I of the user U by directly receiving, from the cloud server 300, the personalized information I of the user U that has been generated or enhanced based on the real-life routine(s) F and stored in the database 310. In which, the received personalized information I may be generated or enhanced by the artificial intelligence module 320 by the cloud server 300 in advance or in response to the request from the robotic device 100. In other embodiments, the processing unit 110 may provide the personalized information I of the user U based on data from the robotic device 100 itself by, for example, directly receiving, from a database in the storage unit 120 of the robotic device 100, the personalized information I of the user U that is generated or enhanced based on the real-life routine(s) F of the user U.

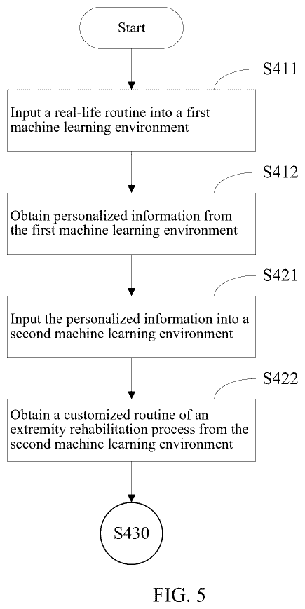

FIG. 5 is a flow chart of an example of generating a customized routine C of an extremity rehabilitation process E (not shown) according to some embodiments of the present disclosure. As shown in FIG. 5, the personalized information I of the user U and the customized routine C of the extremity rehabilitation process E may be generated through machine learning means provided by the artificial intelligence module 320 of the cloud server 300. In some embodiments, the personalized information I is generated in an initiative manner by the artificial intelligence module 320, and the customized routine C is generated in a passive manner in response to the request from the robotic device 100. Accordingly, at step S411, the artificial intelligence module 320 inputs the real-life routine F of the user U into a first machine learning environment (e.g., a first machine learning model). At step S412, the artificial intelligence module 320 obtains the personalized information I of the user U from the first machine learning environment. To enhance the efficiency of machine learning, the artificial intelligence module 320 may obtain the personalized information I of the user U by inputting, in addition to the real-life routine F, other information of the user U, for example, rehabilitation information and medical records of the user U, scheduled prescriptions (e.g., specific items provided by the medical personnel of the user U), or a mixture of them. The cloud server 300 may store the obtained personalized information I of the user U in the database 310, so that the robotic device 100 may request the stored personalized information I from the cloud server 300.

The processing unit 110 may further generate the customized routine C of the extremity rehabilitation process E based on the personalized information I of the user U (block 420 of FIG. 4). Each customized routine C corresponds to one real-life routine F (e.g., a wake-up routine), which includes a plurality of customized tasks (e.g., brushing, bathing, and wearing cloth) and rule(s) (e.g., a sequence of brushing, bathing, and wearing cloth) generated based on the personalized information I of the user U. The customized tasks of each customized routine C may correspond to the above-mentioned real-life items in the real-life routine F, and therefore may be items of daily living activities of the user U like personal hygiene (e.g., brushing, washing face, and bathing), that of personal habits like habitual actions (e.g., wearing eyeglass, wearing cloth, carrying bag), or a mixture of them. The rule of each customized routine C may include the sequence(s) of the customized tasks in the customized routine C, the condition for finishing each customized task, the condition for moving on to the next customized task, or the like.

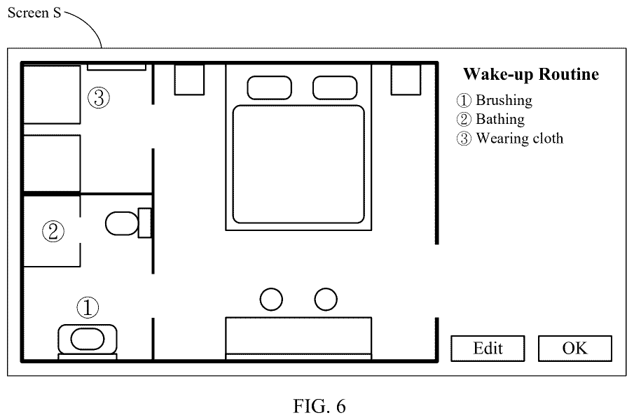

The conducting of the extremity rehabilitation process E may be represented, through the robotic device 100 and the display device 200, as the execution of a game that combines physical and cognitive rehabilitations for robotic therapy, which provides physical rehabilitations through the customized tasks each represented as a part of the game (e.g., brushing, bathing, or wearing cloth), and provides cognitive rehabilitations through the rule that arranges the parts of the game to provide the experience of real-life by, for example, executing the parts of the game with a real-life-like sequence like a wake-up routine of brushing, bathing, and wearing cloth. A game engine may be used to develop the game. FIG. 6 is a schematic diagram of displaying a scene S of a wake-up routine through the display device 200 of the healthcare system 10 of FIG. 2. As shown in FIG. 6, the screen S which includes the scene (i.e., a bedroom with a bathroom and a cloakroom aside) of the wake-up routine is displayed by the display device 200. In the screen S, the right part shows the items of the wake-up routine, namely brushing, bathing, and wearing cloth that are numbered with 1, 2, and 3, respectively, and the left part shows the scene of the wake-up routine that includes the forgoing numbers 1, 2, and 3 indicating the position to perform the corresponding item.

As shown in FIG. 5, at step S421, the personalized information I of the user U is input into a second machine learning environment. At step S422, the customized routine C of the extremity rehabilitation process E is obtained from the second machine learning environment. In some embodiments, the second machine learning environment includes a generative machine learning model, such that by inputting the personalized information I of the user U that is generated based on the real-life routine(s) F of the user U into the generative machine learning model, the customized routine C of the extremity rehabilitation process E is output by the generative machine learning model. In addition, the generated customized routine C may be stored in the storage unit 120 and/or upload to the cloud server 300. Furthermore, the generated customized routine C may also be modified/edited by the user U by, for example, displaying through the display device 200 and modified/edited through the above-mentioned physical button(s) or remote control of the robotic device 100 or the display device 200.

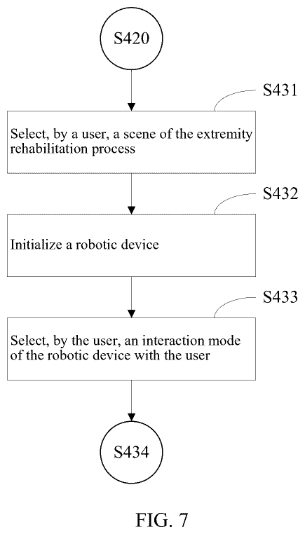

The processing unit 110 may further obtain an interaction result R by conducting the extremity rehabilitation process E according to the customized routine C (block 430 of FIG. 4). According to the upper limb rehabilitation method, before conducting the extremity rehabilitation process E, the processing unit 110 has to perform an initialization process of the extremity rehabilitation process E by determining a scene of the extremity rehabilitation process E and initializing the robotic device 100. FIG. 7 is a flow chart of an example of the initialization process of the extremity rehabilitation process E according to some embodiments of the present disclosure. As shown in FIG. 7, at step S431, the scene of the extremity rehabilitation process E is selected by the user U. Scenes of a room, a space, a floor, a house, or the like may be provided according to, for example, (real-life space information in) the personalized information I of the user U through the display device 200 by displaying as options in textual/graphical manner like a customization screen of the above-mentioned game, so that the user U can select one of the displayed scenes as the scene of the extremity rehabilitation process E through, for example, the above-mentioned physical button(s) or remote control of the robotic device 100 or the display device 200. At step S432, the robotic device 100 is initialized. The robotic device 100 may be initialized by, for example, checking the status of the robotic device 100, establishing connections with parts of the robotic device 100 (e.g., the controller of the motor M), calibrating sensors of the robotic device 100(e.g., the force sensor(s) F) and initializing parts of the robotic device 100 that are related to the customized routine C of the extremity rehabilitation process E. At step S433, an interaction mode of the robotic device 100 with the user U is selected by the user U.

In some embodiments, interaction modes of the robotic device 100 with the user U may be provided through the display device 200 by displaying as options in textual/graphical manner like another customization screen of the above-mentioned game, so that the user U can select one of the displayed interaction modes as the interaction mode (which is equivalent to the mode of the above-mentioned game) of the robotic device 100 through, for example, the above-mentioned physical button(s) or remote control of the robotic device 100 or the display device 200. The interaction modes may include a free mode representing that the user U controls the robotic device 100 without any assistance or force, an assist mode representing that the robotic device 100 provides assistance or force to the user U, and a resist mode representing that the robotic device 100 applies force to the user U so that the user U needs to add strength to control the robotic device 100.

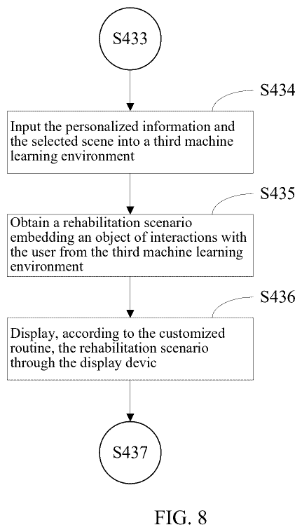

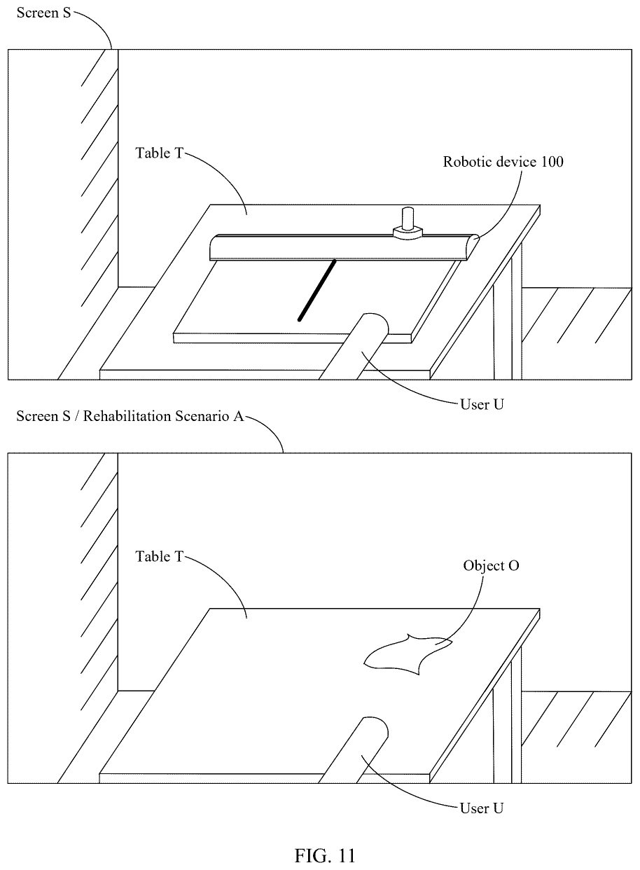

According to the upper limb rehabilitation method, after performing the above-mentioned initialization process of the extremity rehabilitation process E, the processing unit 110 may conduct the extremity rehabilitation process E (which is equivalent to start the above-mentioned game) by displaying object(s) O (see FIG. 11) of interactions N (not shown) with the user U in the selected scene through the display device 200 and controlling the robotic device 100 to perform the interactions N with the user U. In which, each interaction N include physical contact(s) between the user U and robotic device 100 during conducting the extremity rehabilitation process E. The display of the object(s) O may be realized by requesting the cloud server 300. FIG. 8 is a flow chart of an example of displaying the object O of the interactions N in the scene through the display device 200 of the healthcare system 10 of FIG. 2 according to some embodiments of the present disclosure. As shown in FIG. 8, at step S434, the personalized information I and the selected scene are input into a third machine learning environment. At step S435, a rehabilitation scenario A (see FIG. 11) embedding the object O of the interactions N is obtained from the third machine learning environment. At step S436, the rehabilitation scenario A is displayed according to the customized routine C through the display device 200. The third machine learning environment may include a generative machine learning model. In some embodiments, the cloud server 300 may obtain the rehabilitation scenario A through machine learning means provided by the artificial intelligence module 32 and request the display device 200 to display the rehabilitation scenario A according to the customized routine C. The artificial intelligence module 32 may input the personalized information I and the selected scene into the generative machine learning model, such that the generative machine learning model outputs the rehabilitation scenario A that fuses the object O of the interactions N and the selected scene.

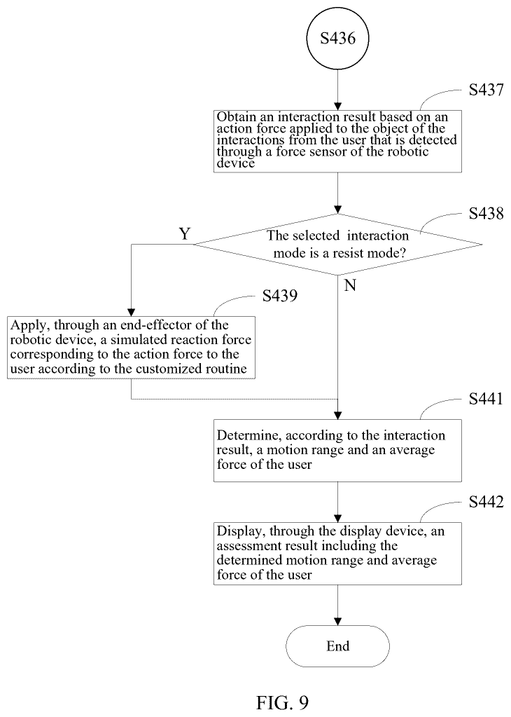

FIG. 9 is a flow chart of an example of controlling the robotic device 100 of FIG. 3 to perform the interactions N with the user U to generating a personalized report P (see FIG. 4) according to some embodiments of the present disclosure. As shown in FIG. 9, at step S437, the interaction result R (i.e., a result of the interactions N) corresponding to the interactions N is obtained based on an action force applied to the object O of the interactions N from the user U, where the action force is detected through a force sensor of the robotic device 100. At step S438, whether the selected interaction mode is the above-mentioned resist mode is determined. At step S439, if yes, a simulated reaction force corresponding to the action force is applied to the user U through an end-effector of the robotic device 100 according to the customized routine C. In which, the simulated reaction force simulates a reaction force that corresponds to the action force applied to the user from the object O. In some embodiments, the force sensor may be a one-axis torque sensor. In which, the end-effector of the robotic device 100 has a plurality of joints each having a motor M, the torque sensor to measure an output torque of the motor M, and an encoder to track a position of the motor M. In other embodiments, the force sensor may be other device such as a torque gauge to measure the output torque. The interaction result R corresponding to the interactions N may be obtained by, for example, recording the output torques of the motor M that are measured by the torque sensor during conducting the extremity rehabilitation process E in a force table of the interaction result R, and recording the positions of the motor M that are tracked by the encoder during conducting the extremity rehabilitation process E in a position table of the interaction result R.

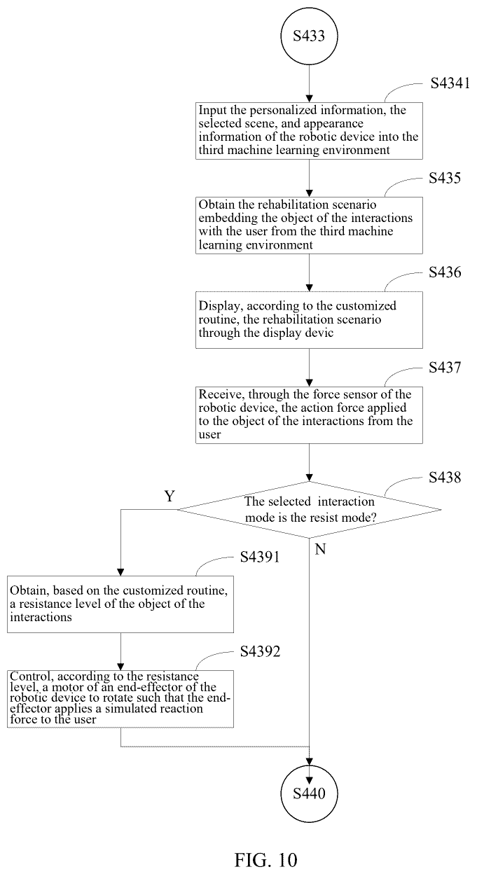

FIG. 10 is a flow chart of an example of conducting the extremity rehabilitation process E in AR manner according to some embodiments of the present disclosure; and FIG. 11 is a schematic diagram of displaying the result of the example of FIG. 10 that conducts the extremity rehabilitation process E in AR manner through the display device 100 of the healthcare system 10 of FIG. 2. The rehabilitation scenario A embedding the object O of the interactions N is displayed through the display device 200, and the robotic device 100 is controlled to perform the interactions N with the user U. As shown in FIG. 10, steps S435, S436, S437, and S438 are the same as that in FIG. 8 and FIG. 9. At step S4341, the personalized information I, the selected scene, and appearance information of the robotic device 100 are input into the third machine learning environment. At step S4391, a resistance level of the object O of the interactions N is obtained based on the customized routine C. At step S4392, the motor M of the end-effector of the robotic device 100 is controlled to rotate according to the resistance level such that the end-effector applies the simulated reaction force corresponding to the action force to the user U. As shown in FIG. 11, the upper part is the screen S displayed by the display device 200 in the above-mentioned real mode that shows a real image of the surroundings of the user U that is captured by the camera of the display device 200. The lower part is the screen S displayed by the display device 200 in the above-mentioned AR mode when conducting the extremity rehabilitation process E in AR manner, which shows the rehabilitation scenario A that fuses the object O and the real image by replacing the robotic device 100 in the real image with the object O (e.g., a wiper) of the interactions N.

The appearance information may include information about the appearance of the robotic device 100 like images of the robotic device 100 or other suitable information that can be used as training data for the third machine learning environment. The resistance level depends on the type of the object O. For example, different objects O such as a wiper and an apple have different resistance levels. The resistance level also depends on the type of the interactions N related to the object O. For example, different related interactions N related to the same objects O like picking up a wiper and using the wiper to wipe the table T have different resistance levels. In some embodiments, the third machine learning environment include a generative machine learning model, such that by inputting the personalized information I, the selected scene, and the appearance information of the robotic device 100 into the generative machine learning model, the rehabilitation scenario A that fuses the object O of the interactions N and the selected scene is output by the generative machine learning model. In addition, the motor M of the end-effector of the robotic device 100 may be controlled to rotate according to the resistance level by obtaining a torque control parameter using a transfer function, where the transfer function is for calculating the torque control parameter corresponding to the resistance level based on a difference between the output torquer and a theoretical torque of the motor M, and controlling the motor M to rotate according to the torque control parameter.

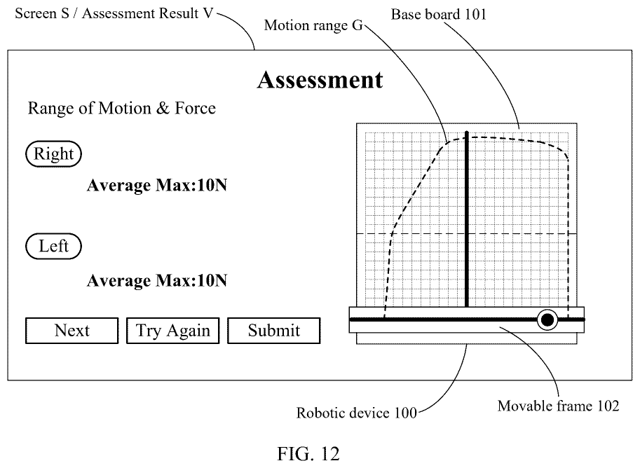

The processing unit 110 may further generate the personalized report P for the user U based on the interaction result(s) R (block 440 of FIG. 4). The personalized report P may include an assessment result V (see FIG. 12). As shown in FIG. 9, at step S441, a motion range G (see FIG. 12) and an average force of the user U is determined according to the interaction result R. At step S442, the assessment result V including the determined motion range G and average force of the user U is displayed through the display device 200. In which, the motion range G is the range of the upper limb of the user U to move upon the base board 101 during conducting the extremity rehabilitation process E. The average force is the average value of the forces of the upper limb of the user U that apply to the robotic device 100 during conducting the extremity rehabilitation process E. In some embodiments, the motion range G may be determined according to, for example, the positions tracked by the above-mentioned encoder of the joint of the robotic device 100 that are recorded in the above-mentioned position table of the interaction result R. The average force may be determined according to, for example, the output torques measured by the above-mentioned torque sensor of the joint of the robotic device 100 that are recorded in the above-mentioned force table of the interaction result R. FIG. 12 is a schematic diagram of displaying the result of the example of FIG. 9 that generates the personalized report R for the user U through the display device 200 of the healthcare system 10 of FIG. 2. As shown in FIG. 12, the screen S which includes the assessment result V is displayed by the display device 200 in response to, for example, the termination of the extremity rehabilitation process E (which is equivalent to the end of the above-mentioned game) or a request from the user U. In the assessment result V, the left part shows the average forces of the right arm and the left arm of the user U, and the right part shows the motion range G of the right arm of the user U (the motion range G is enclosed by the dashed line and the movable frame 102 shown in the assessment result V).

It can be understood by those skilled in the art that, all or part of the method in the above-mentioned embodiment(s) can be implemented by one or more computer programs to instruct related hardware. In addition, the one or more programs can be stored in a non-transitory computer readable storage medium. When the one or more programs are executed, all or part of the corresponding method in the above-mentioned embodiment(s) is performed. Any reference to a storage, a memory, a database or other medium may include non-transitory and/or transitory memory. Non-transitory memory may include read only memory (ROM), programmable ROM (PROM), electrically programmable ROM (EPROM), electrically erasable programmable ROM (EEPROM), flash memory, solid-state drive (SSD), or the like. Volatile memory may include random access memory (RAM), external cache memory, or the like.

The processing unit 110 (and the above-mentioned processor) may include central processing unit (CPU), or be other general purpose processor, graphics processing unit (GPU), digital signal processor (DSP), application specific integrated circuit (ASIC), field-programmable gate array (FPGA), or be other programmable logic device, discrete gate, transistor logic device, and discrete hardware component. The general purpose processor may be microprocessor, or the processor may also be any conventional processor. The storage unit 120 (and the above-mentioned memory) may include internal storage unit such as hard disk and internal memory. The storage unit 120 may also include external storage device such as plug-in hard disk, smart media card (SMC), secure digital (SD) card, and flash card.

The exemplificative units/modules and methods/steps described in the embodiments may be implemented through software, hardware, or a combination of software and hardware. Whether these functions are implemented through software or hardware depends on the specific application and design constraints of the technical schemes. The above-mentioned path planning method and mobile machine may be implemented in other manners. For example, the division of units/modules is merely a logical functional division, and other division manner may be used in actual implementations, that is, multiple units/modules may be combined or be integrated into another system, or some of the features may be ignored or not performed. In addition, the above-mentioned mutual coupling/connection may be direct coupling/connection or communication connection, and may also be indirect coupling/connection or communication connection through some interfaces/devices, and may also be electrical, mechanical or in other forms.

The above-mentioned embodiments are merely intended for describing but not for limiting the technical schemes of the present disclosure. Although the present disclosure is described in detail with reference to the above-mentioned embodiments, the technical schemes in each of the above-mentioned embodiments may still be modified, or some of the technical features may be equivalently replaced, so that these modifications or replacements do not make the essence of the corresponding technical schemes depart from the spirit and scope of the technical schemes of each of the embodiments of the present disclosure, and should be included within the scope of the present disclosure.

What is claimed is:

1. A method for assisting a user to perform extremity rehabilitation using a robotic device and a display device, comprising:
receiving, from a database, personalized information of the user, wherein the personalized information is generated based on at least a real-life routine of the user;
generating, based on the personalized information, at least a customized routine of an extremity rehabilitation process;
determining a scene of the extremity rehabilitation process;
conducting the extremity rehabilitation process by, according to the customized routine, controlling the robotic device to perform interactions with the user and displaying at least an object of the interactions in the scene through the display device; and
generating, based on a result of the interactions, a personalized report for the user.

2. The method of claim 1, wherein the real-life routine includes a plurality of real-life items selected from daily living activities or personal habits of the user.

3. The method of claim 1, wherein generating, based on the personalized information, the customized routine of the extremity rehabilitation process comprises:
inputting the personalized information into a machine learning environment; and
obtaining the customized routine of the extremity rehabilitation process from the machine learning environment.

4. The method of claim 3, wherein the machine learning environment includes a generative machine learning model; inputting the personalized information into the machine learning environment comprises:
inputting the personalized information into the generative machine learning model; and
obtaining the customized routine of the extremity rehabilitation process from the machine learning environment comprises:
obtaining the customized routine of the extremity rehabilitation process from the generative machine learning model.

5. The method of claim 1, wherein controlling the robotic device to perform the interactions with the user comprises:
detecting, through one or more force sensors of the robotic device, an action force applied to the object of the interactions from the user; and
before generating, based on the result of the interactions, the personalized report for the user, the method further comprises:
obtaining, based on the received action force, the result of the interaction.

6. The method of claim 5, wherein before conducting the extremity rehabilitation process, the method further comprises:
selecting, by the user, an interaction mode of the robotic device with the user; and before detecting the action force applied to the object of the interactions from the user, controlling the robotic device to perform the interactions with the user further comprises:
applying, through an end-effector of the robotic device, a simulated reaction force corresponding to the action force to the user according to the customized routine, in response to the selected interaction mode being a resist mode.

7. The method of claim 6, wherein the force sensor is a one-axis torque sensor, and the end-effector of the robotic device has a plurality of joints each having a motor and the torque sensor to measure an output torque of the motor; applying, through the end-effector of the robotic device, the simulated reaction force corresponding to the action force to the user according to the customized routine comprises:
obtaining, based on the customized routine, a resistance level of the object of the interactions; and
controlling, according to the resistance level, the motor of the end-effector of the robotic device to rotate such that the end-effector applies the simulated reaction force corresponding to the action force to the user.

8. The method of claim 7, wherein controlling the motor of the end-effector of the robotic device to rotate according to the resistance level comprises:
obtaining a torque control parameter using a transfer function, wherein the transfer function is for calculating the torque control parameter corresponding to the resistance level based on a difference between the output torquer and a theoretical torque of the motor of the end-effector of the robotic device; and
controlling the motor to rotate according to the torque control parameter.

9. The method of claim 1, wherein displaying the object of the interactions in the scene through the display device comprises:
inputting the personalized information and the determined scene into a machine learning environment;
obtaining a rehabilitation scenario embedding the object of the interactions from the machine learning environment; and
displaying, according to the customized routine, the rehabilitation scenario through the display device.

10. The method of claim 9, wherein the machine learning environment includes a generative machine learning model; inputting the personalized information and the determined scene into a machine learning environment comprises:
inputting the personalized information and the determined scene into the generative machine learning model; and
obtaining a rehabilitation scenario embedding the object of the interactions from the machine learning environment comprises:
obtaining the rehabilitation scenario embedding the object of the interactions from the generative machine learning model.

11. The method of claim 9, wherein the object of the interactions is a simulated object, inputting the personalized information and the determined scene into the machine learning environment comprises:
inputting the personalized information, the determined scene, and appearance information of the robotic device into the machine learning environment; and
obtaining the rehabilitation scenario embedding the object of the interactions from the machine learning environment comprises:
obtaining the rehabilitation scenario embedding the simulated object from the machine learning environment.

12. The method of claim 1, wherein the personalized information is generated by:
inputting the real-life routine into a machine learning environment; and
obtaining the personalized information from the machine learning environment.

13. The method of claim 1, wherein the personalized report includes an assessment result; generating, based on the result of the interactions, the personalized report for the user comprises:
determining, according to the result of the interactions, at least one of a motion range and an average force of the user; and
displaying, through the display device, the assessment result including the determined at least one of the motion range and the average force of the user.

14. A robotic device, comprising:
one or more force sensors;
one or more processors; and
one or more memories storing one or more programs configured to be executed by the one or more processors, wherein the one or more programs comprise instructions to:
receive, from a database, personalized information of the user, wherein the personalized information is generated based on at least a real-life routine of the user;
generate, based on the personalized information, at least a customized routine of an extremity rehabilitation process;
determine a scene of the extremity rehabilitation process;
conduct the extremity rehabilitation process by, according to the customized routine, detecting an action force applied to at least an object of interactions with the user through the one or more force sensors and displaying the object of the interactions in the scene through a display device;
obtain, based on the received action force, a result of the interaction; and
generate, based on the result of the interactions, a personalized report for the user.

15. The robotic device of claim 14, wherein the real-life routine includes a plurality of real-life items selected from daily living activities or personal habits of the user.

16. The robotic device of claim 14, wherein generating, based on the personalized information, the customized routine of the extremity rehabilitation process comprises:
inputting the personalized information into a machine learning environment; and
obtaining the customized routine of the extremity rehabilitation process from the machine learning environment.

17. The robotic device of claim 16, wherein the machine learning environment includes a generative machine learning model; inputting the personalized information into the machine learning environment comprises:
inputting the personalized information into the generative machine learning model; and
obtaining the customized routine of the extremity rehabilitation process from the machine learning environment comprises:
obtaining the customized routine of the extremity rehabilitation process from the generative machine learning model.

18. The robotic device of claim 17, wherein the one or more programs further comprise instructions to:
select, by the user, an interaction mode of the robotic device with the user; and
apply, through an end-effector of the robotic device, a simulated reaction force corresponding to the action force to the user according to the customized routine, in response to the selected interaction mode being a resist mode.

19. The robotic device of claim 18, wherein the force sensor is a one-axis torque sensor, and the end-effector of the robotic device has a plurality of joints each having a motor and the torque sensor to measure an output torque of the motor; applying, through the end-effector of the robotic device, the simulated reaction force corresponding to the action force to the user according to the customized routine comprises:
obtaining, based on the customized routine, a resistance level of the object of the interactions; and
controlling, according to the resistance level, the motor of the end-effector of the robotic device to rotate such that the end-effector applies the simulated reaction force corresponding to the action force to the user.

20. The robotic device of claim 19, wherein controlling the motor of the end-effector of the robotic device to rotate according to the resistance level comprises:
obtaining a torque control parameter using a transfer function, wherein the transfer function is for calculating the torque control parameter corresponding to the resistance level based on a difference between the output torquer and a theoretical torque of the motor of the end-effector of the robotic device; and
controlling the motor to rotate according to the torque control parameter.
